(19) United States
(12) Patent Application Publication
(10) Pub. No.: US 2026/0224770 A1
(43) Pub. Date: Aug. 6, 2026

(54) Fragrance Dispensing System

(71) Applicant: TANGENT DESIGN & ADVERTISING PTE. LTD, Singapore (SG)

(72) Inventors: Richard TAN, Singapore (SG); Theng LIM, Singapore (SG); Nicholas KAMAL, Singapore (SG)

(21) Appl. No.: 19/530,852

(22) Filed: Feb. 5, 2026

(30) Foreign Application Priority Data
Feb. 6, 2025 (SG) 10202500325P

(51) Int. Cl. A61L 9/12

(52) U.S. Cl. CPC A61L 9/125; A61L 2202/16; A61L 2209/11; A61L 2209/133

(57) ABSTRACT

A fragrance dispensing system includes a fragrance dispensing device equipped with one or more fragrance dispensing units configured for dispensing one or more fragrances stored within the fragrance dispensing units. A mobile device has a mobile application with one or more user interfaces. The mobile device is configured for providing a set of fragrance dispensing instructions that correspond to the dispensing of fragrances or fragrance mixtures from the fragrance dispensing device. A storage module is in wireless communication with the fragrance dispensing device and the mobile device, configured for storing data corresponding to the fragrances. The fragrance dispensing system accesses the stored data in the storage module and incorporates machine intelligence to aggregate the data corresponding to the fragrances and fragrance mixtures into predetermined fragrance categories and provide recommendations of different fragrance mixtures which complement user requirements and preferences.

### CROSS-REFERENCE TO RELATED APPLICATIONS

This application claims priority to, and the benefit of, Singapore Provisional Patent Application No. 10202500325P, filed Feb. 6, 2025. The contents of that application are incorporated by reference herein in their entirety.

### TECHNICAL FIELD

The present invention relates to a fragrance dispensing system. More particularly, the present invention relates to a fragrance dispensing system that aggregates data on customized blends of fragrances to be recommended to users based on their specific requirements and preferences.

### BACKGROUND

A sensory experience, particularly olfactory experience, is an experience had by a person when perceiving one or more scent or fragrance stimuli, such as mixture and/or series of fragrances, and can be either a conscious or sub-conscious experience. For a user to perceive this fragrance, its molecules must reach the user's olfactory receptors, which may be achieved through static delivery methods or dynamic diffusion methods. Many types of fragrance dispensing devices have been invented over the years such as passive devices that passively diffuse fragrance substances in a liquid state or a solid state, and active devices that actively diffuse the fragrance substances. However, conventional devices either automatically diffuse the fragrance substances periodically or diffuse the fragrance substances based on a detection of a surrounding environment. Therefore, conventional fragrance devices may be hard to control especially for the intensity and mixtures of the fragrances based on the user's preferences.

Many technologies have been implemented to improve on such a fragrance dispensing system. For example, a United States patent with publication no. US20140365601A1 discloses a system or process for communication, and emission, of scent, by means of a standalone or networked computer, a phone, a scent-device, and a mobile personal scent device, in which the system or process enables communication of additional elements of scent, and communication and emission of scent by means of a scent-device.

Another United States patent with publication no. US20210052762A1 discloses an aroma diffusing system comprising an aroma diffusing device and a mobile device, whereby the aroma diffusing device includes a wireless communication module. The aroma diffusing device is equipped with first through n-th aroma capsules selected among a plurality of aroma capsules, which emit respective aromatic substances having different scents from each other. The mobile device is connected to the aroma diffusing device through a wireless communication. The mobile device independently controls an intensity of the emission of the aromatic substance of each of the first through n-th aroma capsules, which are installed in the aroma diffusing device, to determine a combination of scents diffused from the aroma diffusing device. The mobile device controls the intensity of the emission of the aromatic substance of each of the first through n-th aroma capsules by digitizing the intensity to an integer value.

Yet another United States patent with publication no. US20230375998A1 recites a method for controlling one or more scent delivery units including maintaining one or more scheduled events, maintaining one or more scheduled anti-events, and generating, based on the one or more scheduled events and the one or more scheduled anti-events, command data to be communicated to the one or more scent delivery units to control their activation and deactivation. Generating the command data includes identifying a conflicting period of time during which control specified by the one or more scheduled events differs from control specified by the one or more scheduled anti-events and also includes generating command data that gives priority to control specified by the one or more scheduled anti-events. Control for the one or more scent delivery units during the conflicting period of time is in accordance with control logic of the one or more scheduled anti-events and not the one or more scheduled events.

Lastly, a United States patent with publication no. US20160231720A1 discloses a customized scent diffusing device and a customized diffusing method for effectively transferring sensibility which can be felt from contents to a user by diffusing a most appropriate scent by synchronization with digital contents. According to various exemplary embodiments of the present invention, although odor effect information in 4D contents does not correspond to a spice cartridge configuration in a scent diffusing device, an appropriate odor can be provided through combining or mixing spice cartridges in the scent diffusing device.

### BRIEF SUMMARY

An object of the present invention is to provide a system that allows users to choose between predetermined fragrance mixtures or fully customizing their own blend of fragrance mixtures with supplement from machine intelligence or artificial intelligence. Another object of the present invention is to allow users to share and recommend their fragrance mixtures to a community of users. It is also an object of the present invention to provide a system that can provide one or more recommendations of fragrances and fragrance mixtures based on not only their usage history, but also their activities and preferences, and from fragrances and fragrance mixtures recommended from the community of users.

In one aspect of the present invention, there is provided a fragrance dispensing system, the system comprising a fragrance dispensing device equipped with one or more fragrance dispensing units, configured for dispensing one or more fragrances stored within the fragrance dispensing units; a mobile device having a mobile application with one or more user interfaces, the mobile device being configured for providing a set of fragrance dispensing instructions that correspond to the dispensing of fragrances or fragrance mixtures from the fragrance dispensing device; and a storage module in wireless communication with the fragrance dispensing device and the mobile device, configured for storing data corresponding to the dispensed fragrances; wherein the fragrance dispensing system is further configured to access the stored data in the storage module (300) and incorporates machine intelligence to aggregate the data corresponding to the dispensed fragrances into predetermined fragrance categories and provide recommendations of different fragrance mixtures which complements user requirements and preferences, such that the data corresponding to the fragrances and fragrance mixtures are further incorporated into generating a predetermined media sequence (704), a user-defined media sequence (703), a machine intelligence-defined media sequence, or a combination thereof to be utilized through mobile application (400) thereafter.

Preferably, the fragrance dispensing device is equipped with a wireless communications module configured for enabling wireless communication between the fragrance dispensing device, the mobile device, and the storage module respectively.

Preferably, each of the fragrance dispensing units is equipped with an identifier tag comprising information corresponding to the fragrances within said fragrance dispensing units.

Preferably, the information contained in the identifier tag includes name and type of fragrance stored within the fragrance dispensing unit, configuration settings of the device, a liquid level of the fragrance remaining within the fragrance dispensing unit, or a combination thereof.

Preferably, the user interface is further configured for displaying information of the fragrance dispensing units based on the information contained in the identifier tag.

Preferably, the user interface is further configured for allowing a user to provide a set of input parameters that define a desired sensory experience.

Preferably, the set of input parameters indicate one or more properties of the desired sensory experience selected from a group comprising one or more desired fragrances or fragrance mixtures, one or more temporal properties, one or more desired intensity values, one or more desired fragrance categories, or a combination thereof.

Preferably, the temporal properties include any one or a combination of a start time, an end time, a duration of dispensing, and a repetition frequency.

Preferably, the mobile device includes a processing module that is configured for converting the set of input parameters into the set of fragrance dispensing instructions to be executed by the fragrance dispensing device.

Preferably, the processing module is further configured for receiving user input including voice command or text input to be processed as an information query, and retrieve information from the internet corresponding to the information query to be output to the user interface thereafter.

One skilled in the art will readily appreciate that the present invention is well adapted to carry out the objects and obtain the ends and advantages mentioned, as well as those inherent therein. The embodiment described herein is not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 is a block diagram illustrating a preferred embodiment of a fragrance delivery system.

FIG. 2 is a flowchart illustrating an exemplary embodiment of an overview method for operating the fragrance delivery system.

FIG. 3A illustrates an exemplary embodiment of a user interface displaying a home screen for a mobile application incorporated into a mobile device.

FIG. 3B illustrates an exemplary embodiment of the user interface displaying a user-defined fragrance mixture operation.

FIG. 3C illustrates an exemplary embodiment of the user interface displaying a user-defined media sequence containing user-defined fragrances or fragrance mixtures.

FIG. 3D illustrates an exemplary embodiment of the user interface displaying one or more fragrance categories for available fragrances within a fragrance dispensing device.

FIG. 3E illustrates an exemplary embodiment of the user interface displaying a dispensing operation for a particular fragrance or fragrance mixture.

FIG. 3F illustrates an exemplary embodiment of the user interface displaying a timer during dispensing operation.

FIG. 3G illustrates an exemplary embodiment of the user interface displaying a social network service platform for sharing fragrances or fragrance mixtures.

FIG. 3H illustrates an exemplary embodiment of the user interface displaying a questionnaire for determining and curating recommendations of fragrance or fragrance mixtures to the user.

### DETAILED DESCRIPTION

Hereinafter, the invention shall be described according to the preferred embodiments of the present invention and by referring to the accompanying description and drawings. However, it is to be understood that limiting the description to the preferred embodiments of the invention is merely to facilitate discussion of the present invention and it is envisioned that those skilled in the art may devise various modifications without departing from the scope of the appended claim.

The invention will now be described in greater detail, by way of example, with reference to the drawings.

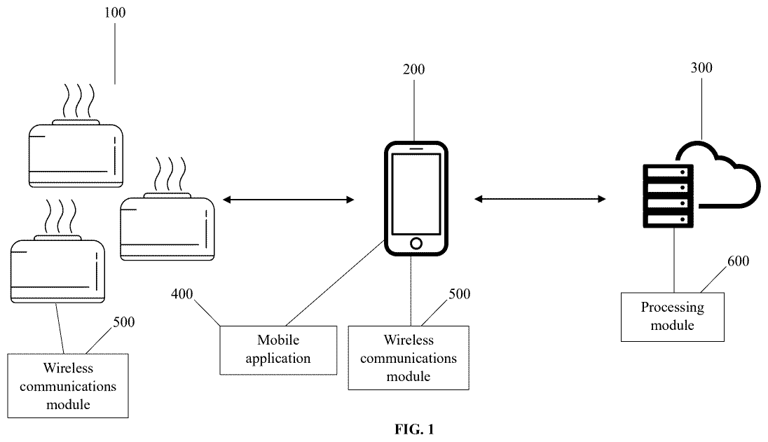

FIG. 1 illustrates a preferred embodiment of a fragrance dispensing system comprising a fragrance dispensing device 100 in wireless communication with a mobile device 200 and a storage module 300 respectively. The communication between the fragrance dispensing device 100 and the mobile device 200 may be by way of signal exchange using wireless communication modules 500 incorporated into the fragrance dispensing device 100 and the mobile device 200 respectively. However, example embodiments are not limited thereto. According to example embodiments, the wireless communication may correspond to a Bluetooth™ communication or a Wireless Fidelity (Wi-Fi) connection. In this case, the wireless communication module 500 included in the fragrance dispensing device 100 may correspond to a Bluetooth communication module of the mobile device 200 to form an interconnected communication network with said mobile device 200.

In this embodiment, the fragrance dispensing device 100 may be equipped with one or more fragrance dispensing units, and is configured for dispensing one or more fragrances stored within the fragrance dispensing units. Preferably, each fragrance dispensing unit includes a fragrance cartridge containing liquid fragrances therein, for example, an essential oil or fragrant oil mixture, that emits a desired fragrance for the particular fragrance dispensing unit. Each of the fragrance dispensing units is also equipped with an identifier tag that comprises of information corresponding to the fragrances within said fragrance dispensing units, in which the information may include a name and type of fragrance stored, configuration settings of the fragrance dispensing device 100, a liquid level of the fragrance remaining in the fragrance dispensing unit, or a combination thereof. However, example embodiments of the information in the identifier tag are not limited thereto.

Although the mobile device 200 as illustrated in FIG. 1 is a smartphone, example embodiments are not limited thereto. The mobile device 100 may be any type of mobile device 100, such as a mobile phone, a tablet computer, a personal digital assistant (PDA), a laptop computer, or the likes. Preferably, the mobile device 100 incorporates a mobile application 400 with one or more user interfaces 700 by which a user can provide a set of input parameters that define a desired sensory experience. In this context, the sensory experience is an olfactory experience which is an experience of smelling which encompasses an entire process of perceiving and interpreting scents from fragrances dispensed by the fragrance dispensing device 100, from an initial detection of odor molecules to conscious recognition and emotional response to a particular smell. The user interface may be displayed on any desired and suitable display screen and manipulated by the user using any desired and suitable input device, such as a keyboard, mouse, touch screen, or the likes. In a preferred embodiment, the user interface 500 may be configured to display information of the fragrance dispensing device 100 and fragrance dispensing units based on the information contained in the identifier tags of each fragrance dispensing units as mentioned above.

In an aspect of the present invention, the set of input parameters that define the desired sensory experience may indicate one or more properties of the desired sensory experience selected from a group comprising one or more desired fragrances or fragrance mixtures, one or more temporal properties, one or more desired intensity values, one or more desired fragrance categories 704, or a combination thereof. By way of example, but not limited to, the temporal properties may include any one or a combination of a start time, an end time, a duration of dispensing, and a repetition frequency of the fragrance dispensing device, the repetition frequency indicating an interval between dispensing of the fragrances across a predetermined period. Preferably, the set of input parameters may be converted, by a processing module 600 incorporated into the mobile device 100, into the set of fragrance dispensing instructions to be provided to the fragrance dispensing units.

When performing the above processes, the processing module 600 may make use of the storage module 300 that comprises stored data corresponding to the dispensed fragrances, the fragrance dispensing units, descriptors of the fragrances, user profiles, or the likes. The instructions provided to the fragrance dispensing units are then followed by said fragrance dispensing units to generate a scent stimulus for the sensory experience. In this embodiment, the storage module 300 may be a digital database such as a cloud network server that is configured for storing data corresponding to the fragrances stored in said fragrance dispensing device, allowing for all information to be stored digitally and access remotely by the user at any given time.

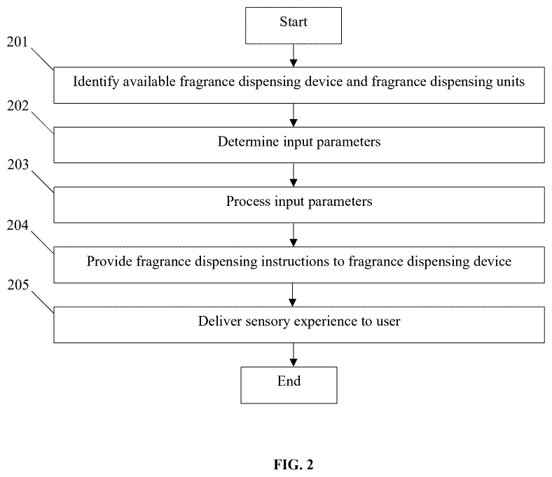

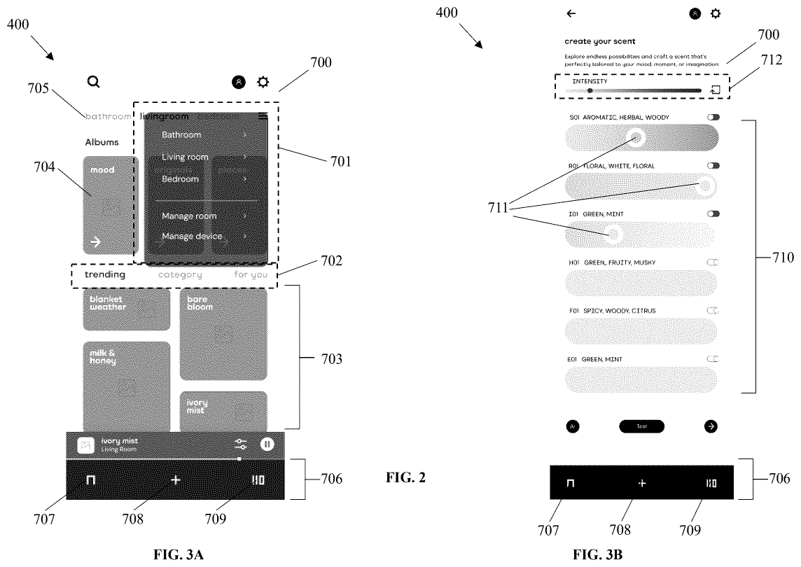

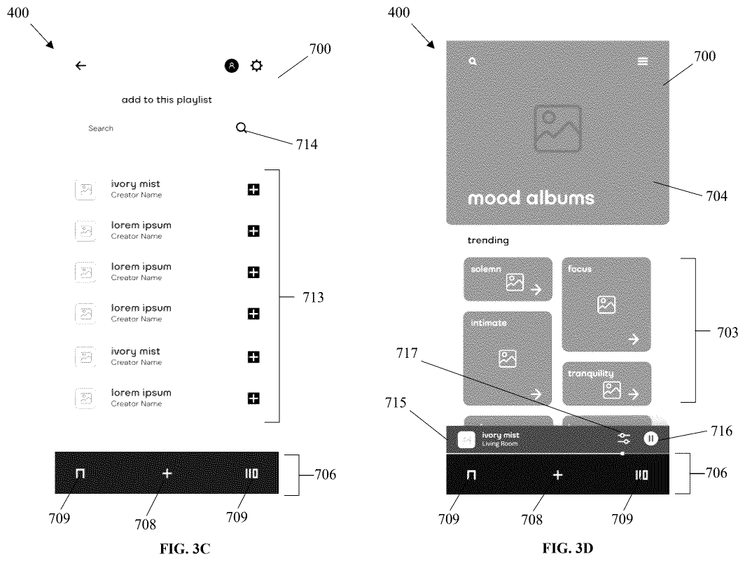

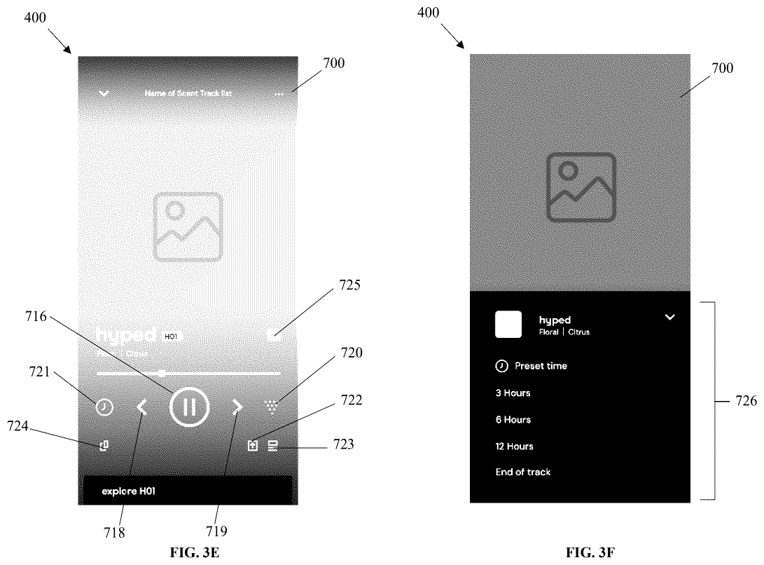

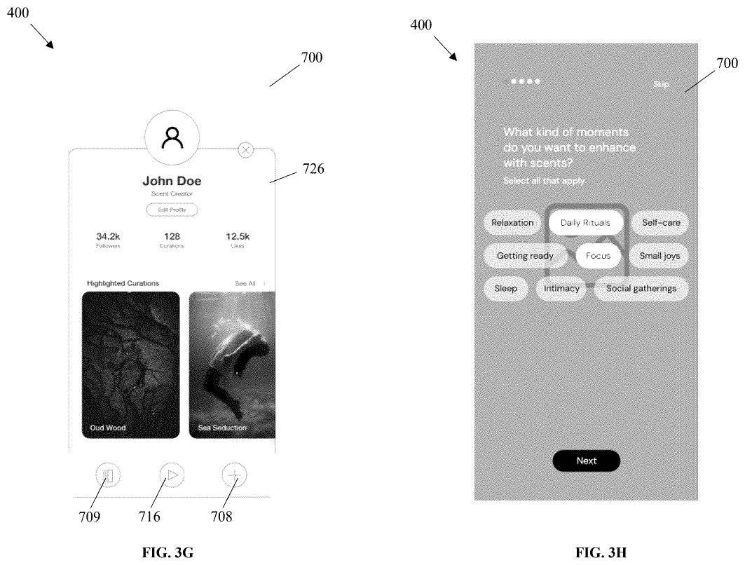

FIG. 2 is a flowchart illustrating an overview method of operation for the fragrance dispensing system. In an embodiment according to Step 201, available fragrance dispensing devices 100 and fragrance dispensing units within said fragrance dispensing devices are firstly identified and reflected within the mobile application 400 of the mobile device 200. Upon identification, the input parameters that define the desired sensory experience may then be determined at Step 202, with the input parameters indicating one or more properties of the desired sensory experience selected from a group comprising one or more desired fragrances or fragrance mixtures, one or more temporal properties, one or more desired intensity values, one or more desired fragrance categories 704, or a combination thereof, as disclosed earlier. At Step 203, the processing module 600 may then process the input parameters to convert the input parameters into fragrance dispensing instructions that is provided to the fragrance dispensing device 100 at Step 204. Once the instructions have been provided to the fragrance dispensing device 100, said fragrance dispensing device 100 may then activate the fragrance dispensing units to dispense the selected fragrances or fragrance mixtures to deliver the sensory experience to the user, as illustrated in Step 205. Further information on the operation of the mobile application 400 will be discussed further herein with reference to FIG. 3A to FIG. 3F.

FIG. 3A to FIG. 3F illustrate various embodiments of the user interfaces 700 of the mobile application 400 for providing the input parameters to achieve the desired sensory experience using the mobile device 200. FIG. 3A illustrates a home screen being displayed on the user interface 700 that can be manipulated by the user. In this embodiment, a top bar 704 may display one or more names of locations corresponding to a position in which the fragrance dispensing devices 100 are disposed in. Upon selecting any one of the locations in the top bar 704, a management tab 701 displaying the location in which the fragrance dispensing device 100 is disposed at, may may be configured as a drop-down menu, such that the user may manipulate the settings of the locations or the fragrance dispensing device 100 if necessary. If there are more than one fragrance dispensing device 100 in a proximity of the user, the user may select between either one fragrance dispensing device 100 to control, or may control all of them simultaneously. Preferably, one fragrance dispensing device 100 may be controlled at one time through the mobile application 200 for convenience.

In a preferred embodiment, the fragrance dispensing system is further configured to access the stored data in the storage module 300 and incorporates machine intelligence or artificial intelligence across the fragrance dispensing device 100, the mobile device 200, and the storage module 300, to aggregate the data corresponding to the fragrances and fragrance mixtures into predetermined fragrance categories 704 and provide recommendations of different fragrance mixtures which complements the user requirements and preferences, such that the data corresponding to the fragrances and fragrance mixtures may then be incorporated to generate a plurality of media sequences 702 which includes a predetermined media sequence, a user-defined media sequence, a machine intelligence-defined media sequence that has personally curated fragrances or fragrance mixtures, or a combination thereof. The plurality of media sequences 702 may be displayed in the form of a vertical carousel, whereby the users may browse through the different media sequences 702 to select the most suitable one.

In particular, the machine intelligence is fed with the input parameters to adapt and learn the usage of the different fragrances for different events, such that the recommended fragrance mixtures are more accurate to the user's requirements and preferences the longer the fragrance dispensing device 100 is in operation. In this embodiment, the event may refer to the type of sensory experience in which the user wants to experience or is experiencing at that given time, such as relaxing, sleeping, reading, working, or the likes. Preferably, the user-defined media sequence may be a selection of fragrances and fragrance mixtures that are personally curated by the user according to the different events, while the predetermined media sequence may be a selection of fragrances and fragrance mixtures that have been pre-selected and input into the mobile application 400 that is displayed according to the available fragrance cartridges within the fragrance dispensing device 100. These media sequences allow the users to experience a different way of selecting fragrances of their choice or those curated based on their requirements and/or preferences.

In a further embodiment, the machine intelligence may be utilized to recognize input prompts such as voice commands or text prompts, whereby the machine intelligence may identify the type of input prompts and processing said input prompts to identify the input parameters to be converted into fragrance dispensing instructions thereafter. Upon identification of input prompts, the machine intelligence may perform a query search over the internet and outputs recommendations of fragrances or fragrance mixtures to be used by the user based on the availability of the different fragrances within the fragrance dispensing device 100. Further, the input prompts may be integrated with Internet-of-Things (IoT) capabilities, such as smart home appliances, where a simple voice command may be used to activate the fragrance dispensing device 100 and the mobile application 200 respectively. In all of the user interfaces 700 illustrated from FIG. 3A to FIG. 3H, a bottom tab bar in all the user interfaces illustrated from FIG. 3A to FIG. 3F, a bottom tab bar 706 may be displayed which may include a “homepage” button 707 that allows the user to return to the home screen from any of the user interfaces 700, an “add” button 708 for including additional fragrances or fragrance mixtures to be dispensed, and a “library” button 709 which displays all the fragrances or fragrance mixtures in queue in the form of a list.

In an exemplary embodiment as illustrated in FIG. 3H, the mobile application 400 may provide a questionnaire to the user that allows the machine intelligence to identify and determine the type of fragrance or fragrance mixtures to be recommended to the user based on the available fragrances in the fragrance dispensing devices 100. One or more sets of questions may be provided to the user, where the user may choose from a selection of options that may be processed thereafter and be provided recommendations of fragrances that are displayed in the plurality of media sequences 702.

FIG. 3B illustrates the user interface 700 where the user may designate one or more fragrances to be dispensed, forming a customized fragrance mixture based on the available fragrance dispensing units in the fragrance dispensing device 100. In this embodiment, interactive tabs 710 representing each available fragrances may be displayed with a plurality of nodes 711, in which each node 711 can be manipulated to adjust a relative intensity value for each fragrance to form the customized fragrance mixture. Preferably, all the nodes 711 may be arranged at an initial position on the interactive tabs 710 indicating a predetermined intensity. The user may alter the intensities of the different fragrances by moving the nodes 711 around each interactive tab 710, which either increases or decreases the node's 711 value. These nodes 711 may be manipulated by the user until the user is satisfied with the combination of intensities of the respective fragrances. An overall intensity of the combination of the fragrances may also be altered via an overall intensity tab 712 arranged above the interactive tabs 710.

The mobile device 200 may then memorize these setting values which corresponds to the intensities of the fragrances being dispensed, and stores this information in the storage module 300 to be accessed at any given time. When the fragrance dispensing device 100 is turned on to operate next time, the mobile device 200 may set the memorized setting values, as the intensities of the respective fragrance dispensing units respectively. The user interface 700 as illustrated in FIG. 3B provides a convenient and intuitive way for the user to provide the set of input parameters that define the desired sensory experience over time.

In another embodiment as illustrated in FIG. 3C, the user may create their own user-defined media sequence, where their personalized fragrances or fragrance mixtures may be stored within said user-defined media sequence in the form of a list 713 based on the available fragrances in the fragrance dispensing device 100. The user may select through the list 713 or type out the name of the fragrance or fragrance mixtures using a search button 714 arranged above the list 713. Preferably, the information corresponding to the user-defined media sequence may also be stored in the storing module 300 to be accessed by the user at any given time.

In an embodiment as illustrated in FIG. 3D, the user can view the different fragrance categories 704 such as, by way of example, moods, places, moments, or trending, to name a few. The different fragrance categories 704 displayed may have one or more fragrances or fragrance mixtures that have been predetermined based on the types of fragrance that are present in the fragrance dispensing device 100. Alternatively, the user may access their user-defined media sequence that contains the saved setting values of the respective customized fragrance mixtures. Additionally, at any time during operation, a pop-up 715 may be displayed showing the selected fragrances or fragrance mixtures that are being dispensed, whereby the pop up 715 may include a pause button 716 that can cease the operation any time, and an edit button 717 that allows the user to configure the settings for dispensing of said fragrances or fragrance mixtures.

FIG. 3E further illustrates the user interface 700 during the dispensing operation of the fragrance or fragrance mixtures as set by the user. The user interface 700 may be designed similarly with conventional music players, where the user may use the pause button 716 to start and stop the fragrance dispensing device 100. The user may also skip a currently dispensed fragrance or fragrance mixtures using either a “previous” button 718 or a “next” button 719. In this exemplary embodiment, the user may alter or adjust the intensity of the dispensing operation by clicking on an intensity button 720 that is arranged adjacent to the “next” button 719, and adjust the temporal properties of the dispensing operation by clicking on a timer button 721 that is adjacent to the “previous” button. Disposed below the intensity button 720 are an upload button 722 that allows the user to store the current media sequence within the storage module 300, and a queue button 723 that displays subsequent fragrances or fragrance mixtures that are queued to be dispensed once for when a current fragrance or fragrance mixture has completed the duration of dispensing. Another button disposed below the timer button 721 is a device button 724 that allows the users to manage and select any one fragrance dispensing devices 100 that are connected to the mobile application 400 within the proximity of the user. An “add” button 725 may also be displayed to include additional fragrances or fragrance mixtures to the queue.

Referring to FIG. 3F, the user interface 700 illustrates settings for a timer when the timer button 721 is selected by the user. In this embodiment, one or more temporal properties may be displayed as a pop-up menu 726 when the timer button 721 is clicked. The temporal properties may be manipulated by the user whether to prolong or shorten the duration of dispensing, and determine a start time and end time of the dispensing operation. This way, an associated stimulus for that event is provided to the user at the indicated start time and for the indicated duration of dispensing. These associations may be chosen by the user and/or learned by machine intelligence or by the user over time, wherein different fragrances, or fragrance mixtures for associating other events may also be selected and/or learned as desired.

Referring to FIG. 3G, the user may provide the different fragrant mixtures or user-defined media sequence to be shared with other users. In this embodiment, the user interface may be configured to perform a social network services (SNS) function in which the user may be provided with a profile 726 that can be shared by users on the same platform, in which the user and a plurality of other uses share preferred fragrance mixtures with each other. This way, the plurality of other users may enjoy the shared fragrance mixtures, and the user may try out different fragrance mixtures as recommended and shared by said plurality of other users. As a result, the fragrance dispensing system may provide an increased satisfaction to the user in terms of user experience.

Any one or more of the various functional elements of the technology described herein, such as, for instance, any of the modules or interfaces described above could, where appropriate, communicate remotely, such as over a network, i.e., the Internet or World Wide Web. Alternatively, the functional elements could all be local to one another and/or part of a single device.

The present disclosure includes as contained in the appended claims, as well as that of the foregoing description. Although this invention has been described in its preferred form with a degree of particularly, it is understood that the present disclosure of the preferred form has been made only by way of example and that numerous changes in the details of construction and the combination and arrangements of parts may be resorted to without departing from the scope of the invention.

We claim:

1. A fragrance dispensing system, the system comprising:
a fragrance dispensing device equipped with one or more fragrance dispensing units, configured for dispensing one or more fragrances stored within the fragrance dispensing units;
a mobile device having a mobile application with one or more user interfaces, the mobile device being configured for providing a set of fragrance dispensing instructions that correspond to the dispensing of fragrances or fragrance mixtures from the fragrance dispensing device; and
a storage module in wireless communication with the fragrance dispensing device and the mobile device, configured for storing data corresponding to the fragrances;
wherein the fragrance dispensing system is further configured to access the stored data in the storage module and incorporates machine intelligence to aggregate the data corresponding to the fragrances and fragrance mixtures into predetermined fragrance categories and provide recommendations of different fragrance mixtures which complements user requirements and preferences, such that the data corresponding to the fragrances and fragrance mixtures are further incorporated into generating a predetermined media sequence, a user-defined media sequence, a machine intelligence-defined media sequence, or a combination thereof to be utilized through mobile application thereafter.

2. The fragrance dispensing system according to claim 1, wherein the fragrance dispensing device is equipped with a wireless communications module configured for enabling wireless communication between the fragrance dispensing device, the mobile device, and the storage module respectively.

3. The fragrance dispensing system according to claim 1, wherein each of the fragrance dispensing units is equipped with an identifier tag comprising information corresponding to the fragrances within said fragrance dispensing units.

4. The fragrance dispensing system according to claim 3, wherein the information contained in the identifier tag includes name and type of fragrance stored within the fragrance dispensing unit, configuration settings of the fragrance dispensing device, a liquid level of the fragrance remaining within the fragrance dispensing unit, or a combination thereof.

5. The fragrance dispensing system according to claim 3, wherein the user interface is further configured for displaying information of the fragrance dispensing units based on the information contained in the identifier tag.

6. The fragrance dispensing system according to claim 1, wherein the user interface is further configured for allowing a user to provide a set of input parameters that define a desired sensory experience.

7. The fragrance dispensing system according to claim 6, wherein the set of input parameters indicate one or more properties of the desired sensory experience selected from a group comprising one or more desired fragrances or fragrance mixtures, one or more temporal properties, one or more desired intensity values, one or more desired fragrance categories, or a combination thereof.

8. The fragrance dispensing system according to claim 7, wherein the temporal properties include any one or a combination of a start time, an end time, a duration of dispensing, and a repetition frequency.

9. The fragrance dispensing system according to claim 6, wherein the mobile device includes a processing module that is configured for converting the set of input parameters into the set of fragrance dispensing instructions to be executed by the fragrance dispensing device.

10. The fragrance dispensing system according to claim 9, wherein the processing module is further configured for receiving user input including voice command or text input to be processed as an information query, and retrieve information from the internet corresponding to the information query to be output to the user interface thereafter.
